(19) United States
(12) Patent Application Publication
(10) Pub. No.: US 2025/0339578 A1
(43) Pub. Date: Nov. 6, 2025

(54) POLY(MELAMINE-SILICATE) DERIVATIVES AS BIOCIDAL AGENTS FOR DISINFECTION AND DECONTAMINATION

(71) Applicant: State of Israel, Prime Minister’s Office, Israel Institute for Biological Research, Nes-Ziona (IL)

(72) Inventors: Dana Maxime MIZRAHI, Rishon LeZion (IL); Shay WEISS, Ness-Ziona (IL); Amir BEN-SHMUEL, Rehovot (IL); Victoria NAHUM, Rishon LeZion (IL)

(21) Appl. No.: 18/854,126

(22) PCT Filed: Mar. 19, 2023
(86) PCT No.: PCT/IL2023/050283

§ 371 Date: Oct. 4, 2024

(30) Foreign Application Priority Data
Apr. 14, 2022 (IL) 292287

(51) Int. Cl. A61L 15/44; A61L 15/20

(52) U.S. Cl. CPC A61L 15/44; A61L 15/20; A61L 2300/204; A61L 2300/404; A61L 2300/60

(57) ABSTRACT

The present subject matter relates to poly(melamine-silicate) derivatives as biocidal disinfection and decontamination agents, and to methods for their production and use. More specifically, the subject matter relates to halogenated adducts of poly(melamine-silicate) with metal oxides.

### FIELD OF THE INVENTION

The present invention relates to the field of biocidal agents. Specifically, the invention relates to poly(melamine-silicate) derivatives as biocidal disinfection and decontamination agents, and to methods for their production and use. More specifically, the invention relates to halogenated adducts of poly(melamine-silicate) with metal oxides.

### BACKGROUND OF THE INVENTION

N-halamines have been broadly used as disinfectants in water purification systems and other applications, while employing relative stability of the active halogen bound to nitrogen during storage and its easy release in contact with oxidizable contaminants.

The halamines have also been incorporated into polymers or textiles for use in liquid stream purification or in biocidal coatings. In multiple applications, various systems have been described, including chloramines derived from amines, amides, or imides, optionally with regeneration of the N-halogen bond, for use as antibacterial agents, in pool sanitation, as additives in laundry or dishwater detergents, as decontaminants and protectants against toxic chemicals and warfare agents.

Resistance toward existing biocides among pests, the appearance of new pests, and looming dangers of malicious biological agents aimed at civil populations by rogue regimes and terrorists, present urgent needs for novel biocidal disinfection and decontamination materials. In parallel, however, safety requirements must be taken into account, as some pesticides pose medical risks, usually including cancer or immune disorders. It is therefore an object of this invention to provide chemical structures which will efficiently but also safely reduce or eliminate deleterious biological activities of agents selected from viruses, bacteria, fungi, protozoa, algae, and parasites.

It is another object of this invention to provide a biocidal material capable to deactivate a pathogen selected from viruses, bacteria, fungi, protozoa, algae, parasites, and toxic organic agents.

It is a further object of this invention to provide a biocidal decontamination material for reducing or eliminating the deleterious biological activity of an agent selected from viruses, bacteria, fungi, protozoa, algae, parasites or eggs thereof, and toxic organic agents including peptides, proteins, bacterial or fungal or algal toxins.

This invention also aims at providing a method of preparing a biocidal decontamination material for reducing or eliminating the deleterious biological activity of an agent selected from viruses, bacteria, fungi, protozoa, algae, parasites or eggs thereof, and toxic organic agents selected from peptides, proteins, lipids, liposaccharides, bacterial or fungal or algal toxins, comprising steps of i) complexing a metal oxide with a melamine-silicate either before or after polymerizing said melamine silicate, and ii) halogenating said melamine-silicate metal complex.

It is also an object of this invention to provide a system for reducing or inhibiting deleterious activity of biological agents selected from viruses, bacteria, fungi, protozoa, algae, parasites or eggs thereof, and toxic organic agents selected from peptides, proteins, lipids, liposaccharides, bacterial or fungal or algal toxins, comprising a complex of metal oxide with poly(melamine-silicate), and chlorinating said complex, optionally repeatedly.

It is another object of the invention to provide novel biocidal disinfection and decontamination agents that may be advantageously used as additives in various chemical compositions.

It is a further object of the invention to provide processes for manufacturing novel compounds active as biocidal disinfection and decontamination agents as well as methods of using the same.

It is also an object of the invention to incorporate the described biocidal disinfection and decontamination materials within polymer matrices, construction materials or textiles.

Still another object of the invention is to use the specified biocidal disinfection and decontamination materials in water purification systems, comprising columns, filters, or coatings.

The invention also aims at using the specified biocidal disinfection and decontamination materials in air purification systems.

Also included in the invention is the optional regeneration of the N-halogen bond, preferably N—Cl bond, after oxidation of the deleterious material.

Other objects and advantages of present invention will appear as the description proceeds.

### SUMMARY OF THE INVENTION

This invention provides a biocidal disinfection and decontamination material, and nonhalogenated precursors thereof, comprising halogenated adduct of a metal oxide with a poly(melamine-silicate), having the following structure:

—[(O . . . M . . . O)—Si-(alkylen-N(X)-2-yl-4-N(X₂)-6-N(X₂)-1,3,5-triazine)-O—]ₙ,
wherein O . . . M . . . O symbolizes adduct of metal oxide with silicate, the bracketed member
[( )Si-( )-O—]ₙ stands for n-times repeated unit of substituted silica in the polymer, X stands for halogen, and M stands for metal, and wherein said alkylene is selected from C₂ to C₈-alkylene, and said metal is selected from Mg, Zn, Ca, Fe, Cu, Ti, Ag, and Al.

In one embodiment of the invention, the biocidal material has the following structure:

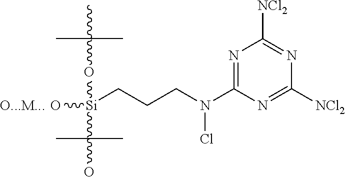

wherein said metal is selected from Mg, Zn, Ca, Fe, Cu, Ti, Ag, or Al.

The invention also relates to a precursor of said biocidal disinfection and decontamination material, enabling to prepare the strongly oxidizing biocide of the invention when needed in situ, without eventual losses of activity during storage. The precursor is an adduct of a metal oxide, such as MgO, ZnO, CaO, Fe₂O₃, Cu₂O, CuO, TiO₂, Al₂O₃, Ag₂O, and AgO, with a poly(melamine-silicate), having the following structure:

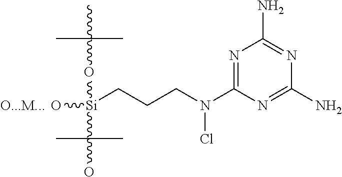

where M is Mg, Zn, Ca, Fe, Cu, Ti, Ag, or Al. It is also a part of the invention, providing an easily storable precursor which can be converted to said biocidal material when needed by halogenation with NaOCl or bromine.

The invention provides a process for manufacturing an efficiently and safely applicable biocidal disinfection and decontamination material, comprising steps of: i) reacting ω-aminoalkyl trialkoxysilane with 2-chloro-4,6-diamino-1,3,5-triazine, thereby providing a melamine-silicate based monomer of the following structure:

2-NH-alkyltrialkoxysilyl-4-NH₂-6-NH₂-1,3,5-triazine

wherein said alkyl is selected from C2 to C8-alkyl, and said alkoxy is selected from C1 to C4-alkoxy; ii) complexing said monomer with said metal oxide and then polymerizing said monomer, or polymerizing said monomer and then complexing the product with said metal oxide, the two ways providing two different adducts with different properties; and iii) halogenating the adduct of step ii) with NaOCl or bromine. In a first aspect of the invention, the process comprises steps of: i) reacting 3-aminopropyl triethoxysilane with 2-chloro-4,6-diamino-1,3,5-triazine, thereby providing a melamine-silicate based monomer of the following structure: 2-aminoalkyltriethoxysilyl-4,6-diamino-1,3,5-triazine; ii) complexing said monomer with said metal oxide and then polymerizing said monomer, thereby obtaining a polymer adduct; and iii) halogenating the adduct of step ii) with NaOCl or NaOBr. In a second aspect of the invention, the process comprises steps of: i) reacting 3-aminopropyl triethoxysilane with 2-chloro-4,6-diamino-1,3,5-triazine, thereby providing a melamine-silicate based monomer of the following structure: 2-aminoalkyltriethoxysilyl-4,6-diamino-1,3,5-triazine; ii) polymerizing said monomer and then complexing the product with said metal oxide, thereby obtaining a polymer adduct; and iii) halogenating the adduct of step ii) with NaOCl or bromine. Said metal may be selected from Mg, Zn, Al, Ag, Fe, Cu, and Ti. Said step of polymerization in the process of the invention comprises heating and distilling out the released alcohol, usually ethanol.

In one preferred aspect, the invention provides a process for efficiently and safely destroying or neutralizing a harmful biological agent on a surface or in a volume, the agent selected from viruses, bacteria, fungi, protozoa, algae, and parasites, comprising contacting said surface or volume with the biocidal disinfection and decontamination material comprising halogenated adduct of a metal oxide with a poly(melamine-silicate) having structures as specified above.

The invention relates to a process for decontaminating a surface or a volume contaminated with a biological agent sensitive to oxidizers, comprising i) complexing a metal oxide with a melamine-silicate either before or after polymerizing said melamine silicate, ii) halogenating said melamine-silicate metal complex, and iii) contacting said surface or volume with said halogenated complex. Said halogenating preferably comprises chlorinating or brominating. Said chlorinating is in some preferred embodiments optionally applied repeatedly on the same object by applying NaOCl when the oxidizing power of the object has been reduced.

The invention further relates to the use of biocidal disinfection and decontamination materials, or precursors thereof, prepared as specified herein, for reducing or eliminating the deleterious biological activity of any harmful material sensitive to oxidation selected from viruses, bacteria, fungi, protozoa, algae, parasites or eggs thereof, and toxic organic agents including peptides, proteins, lipids, polysaccharides, bacterial or fungal or algal toxins. In one aspect, said use comprises paints, coatings, textiles, plastics, or construction materials. In another aspect, the use comprises health and food facilities. In a still another aspect, the use comprises cleaning means, such as means for purifying or filtering fluids, including liquids or gases. Said means may include filters, such as air filters.

The invention provides a textile product impregnated with the biocidal disinfection and decontamination material, namely the halogenated adduct of a metal oxide with a poly(melamine-silicate) as specified above, for providing protective dressings, surgical drapes, or surgical clothes and other auxiliary textiles needed during medical treatments. The invention further provides a textile product impregnated with said biocidal disinfection and decontamination material for protecting the skin of a subject threatened by a skin microbial infection, or for supporting healing process in the skin of a subject in need of the support. Said textile product may be an active antibacterial dressing for contacting the skin of a subject suffering from skin trauma, such as acute skin injury, chronic skin wound, surgical wound, or chronic skin condition including autoimmune or allergic problems. Said textile product is preferably an active dressing for contacting the skin of a subject suffering from acne.

The invention relates to a mixture of the biocidal disinfection and decontamination material, namely the halogenated adduct of a metal oxide with a poly(melamine-silicate) as specified above, with gel, ointment, or cream, optionally further comprising an agent selected from analgesic, antiallergic, antibiotic, antifungal, anti-inflammatory. Said mixture efficiently serves in contacting the skin of a subject suffering from a skin condition aggravated by microbial infections, wherein said mixture may contact the skin directly of while being impregnated within a textile material.

### BRIEF DESCRIPTION OF THE DRAWINGS

The above and other characteristics and advantages of the invention will be more readily apparent through the following examples, and with reference to the appended drawings, wherein:

FIG. 1. is a reaction scheme; FIG. 1A showing the preparation of a monomer in one aspect of the invention and its polymerization; chlorinating step adding five chlorine atoms is further shown; FIG. 1B schematically shows a step of complexing metal oxide (MO) with melamine-silicate;

FIG. 2. demonstrates two ways of preparing poly(melamine-silicate) metal oxide complex according to several embodiments of the invention, different order of complexation/polymerization steps and different metals provide four different products, which products may be chlorinated immediately, or may be stably stored and chlorinated only later when the detoxification activities are needed;

FIG. 3. shows the dose dependent inhibition of bacterial growth for three different bacteria and several materials (in PEVA disks placed on agar); the materials include raw materials, and products prepared according to embodiments of the invention (see the section Examples);

FIG. 4. is Table 1, listing materials which have been tested; and

FIG. 5. shows the dose inhibition of bacterial growth for three different bacteria and several materials (placed in holes punched in the agar); the materials include products prepared according to embodiments of the invention (see the section Examples).

### DETAILED DESCRIPTION OF THE INVENTION

It has now been found that poly(melamine-silicate) derivatives, when complexed with metal oxides, and when halogenated are advantageous means for neutralizing microbial contaminations.

Processes for manufacturing halogenated poly(melamine-silicate) complexed with metal oxide are provided. In one embodiment, the process according to the invention comprises a step of complexing poly(melamine-silicate) with a metal oxide and halogenating; in another embodiment, the process according to the invention comprises a step of complexing a monomeric melamine-silicate derivative with a metal oxide, polymerizing said silicate and halogenating. The metal of said metal oxide may comprise magnesium, zinc, calcium, iron, copper, titanium, aluminum, and silver. In a preferred embodiment, the halogen of said halogenated poly(melamine-silicate) metal oxide complex of the invention is chlorine or bromine. Said complex is an adduct comprising metal oxide and halogen bound to nitrogen atoms of melamine in said melamine silicate. Said adduct is preferably used in neutralizing or in eliminating microbial pathogens. Said metal oxide may comprise, for example, MgO, ZnO, CaO, Fe₂O₃, Cu₂O, CuO, TiO₂, Al₂O₃, Ag₂O, or AgO.

Biocidal resistance among various pests, particularly antibacterial resistance among numerous bacterial species, becomes a global health problem and also financial burden to the healthcare system. For example, it is reported that more than a half of bacterial infections are resistant to one or more of the antibiotics that are generally used. However, other pests, including viruses or worms and insect parasites, develop resistance too, so that much of what has been attained by modern epidemiology might be endangered without introducing new therapeutic systems against the resistant pests. Novel biocides are urgently needed, and that is one aim of the invention to provide halamines exhibiting novel structures.

In an important aspect of the invention, a hybrid product is provided, combining antimicrobial effects of metal oxides with oxidizing effects of halamines. Disinfectant or antiseptic materials acting in different mechanisms are thus united in one molecular entity. In some embodiments of the invention, the adducts involve synergistic actions of halamine silicates and metal oxides. In a preferred embodiment, said metal oxide in the adducts is a nano-sized metal oxide. In one preferred embodiment of the invention, said hybrid biocidal product comprises chlorinated poly(melamine-silicate) magnesium oxide, or zinc oxide, or calcium oxide, or iron oxide, or copper oxide, or titanium oxide, or aluminum oxide, or silver oxide.

The term “biocides” as used herein relates to materials destroying or neutralizing harmful activities caused by biological agents selected from viruses, bacteria, fungi, protozoa, algae, and parasites, wherein parasites are particularly selected from worms, arthropods, and eggs thereof.

Said biocidal disinfection and decontamination materials of the invention provide a unique biocidal system, insoluble in water, comprising metal atoms adduced to silica moiety and strongly oxidizing halogen atoms reversibly bound to the amino groups of melamine.

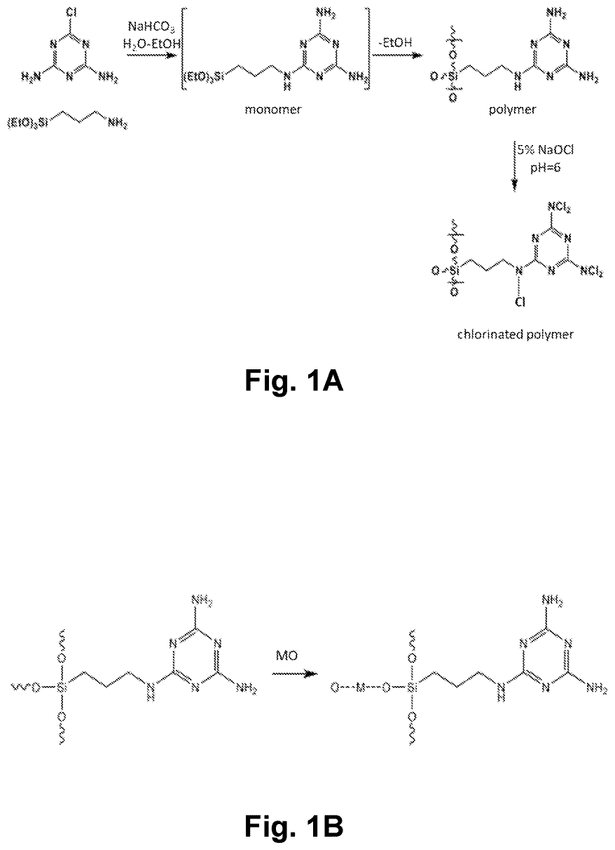

The invention provides a method for preparing poly(melamine silicate) metal oxide complex. In one embodiment of the invention, a process is provided, comprising steps of i) reacting 3-aminopropyl triethoxysilane with 2-chloro-4,6-diamino-1,3,5-triazine, thereby providing 3-melaminepropyltriethoxysilyl (silane-melamine monomer), ii) heating said monomer, thereby condensing the monomer and distilling ethanol out of the mixture, thereby obtaining a poly(melamine-silicate) derivative, namely poly(melamine-propylsilicate). In a preferred embodiment, the process of the invention comprises a step of reacting poly(melamine-silicate) with aqueous hypochlorite, thereby providing chlorinated poly(melamine-silicate) (FIG. 1A). In some embodiments of the invention, metal oxide in a ratio of metal to silicon of 1:1 is mixed with melamine-silicate or poly(melamine-silicate) (FIG. 1B).

Metal oxides were shown to have biocidal activity in an unknown mechanism, possibly due to change in metal concentration, influence on pH or damage to specific structures or processes within bacteria cell. Enhancement of this activity can be achieved by using nanosized metal oxides, thus enlarging the active surface area. Combining two biocidal activities of different mechanisms may result in synergism. The present invention utilizes the silica part of the melamine polymer as the complexation site for metal oxides, providing, together with halogen bound to melamine, a two functional hybrid. Moreover, the structure of the complex according to the invention enables binding up to five halogen atoms per each melamine-silicate monomer (FIG. 1A), providing a very strong oxidizer.

The complex according to the invention can advantageously be incorporated within polymer matrices, construction materials or textiles. Said complex, while halogenated, can deactivate a pathogen selected from viruses, bacteria, fungi, protozoa, algae and parasites. Said complex of the invention may be advantageously used as an additive in various chemical compositions. Said complex can be used in water purification systems, in columns, in filters, in air filters, or in coatings. Said complex comprises N-halogen bonds, preferably N—Cl bonds, which enable oxidizing deleterious materials, and which may be regenerated after releasing said halogen during said oxidizing step.

In one important aspect, the halogenated adduct of a metal oxide with a poly(melamine-silicate) according to the invention is incorporated into a textile material for protecting the environment for medical treatments. In a preferred embodiment, the halogenated adduct of a metal oxide with a poly(melamine-silicate) according to the invention is incorporated into a textile material for providing protective dressings, surgical drapes, or surgical clothes and all needed auxiliary textiles.

In an important aspect of the invention, a textile material impregnated with the halogenated adduct of a metal oxide with a poly(melamine-silicate) is employed to protect the mammalian skin from a microbial threat or for supporting healing process in the skin. In a preferred embodiment, the invention provides an active antibacterial dressing to contact the skin of a subject suffering from skin trauma, such as acute skin injury, chronic skin wound, surgical wound, or chronic skin condition including autoimmune or allergic problems.

In a preferred embodiment, the invention provides a novel treatment for acne. Acne is a chronic, inflammatory condition affecting a great part of the population at some time, resulting in pustules and scars; the existing treatments often include irritation and other harmful effects, so that new treatments are urgently needed. The invention provides a biocidal cloth impregnated with halogenated adduct which efficiently enhances the skin healing processes when applied onto the skin, for example during sleep. The cloth or impregnated dressing or other textile material comprises, in one embodiment of the invention, the halogenated adduct of a metal oxide with a poly(melamine-silicate) and optionally additional active agents selected from analgesic, antiallergic, antibiotic, antifungal, anti-inflammatory. The biocidal cloth of the invention optionally further comprises a carrier of the halogenated adduct selected from gel, ointment, or cream. In another embodiment, gel, ointment, or cream may be directly combined with said halogenated complex and optionally additional active agents.

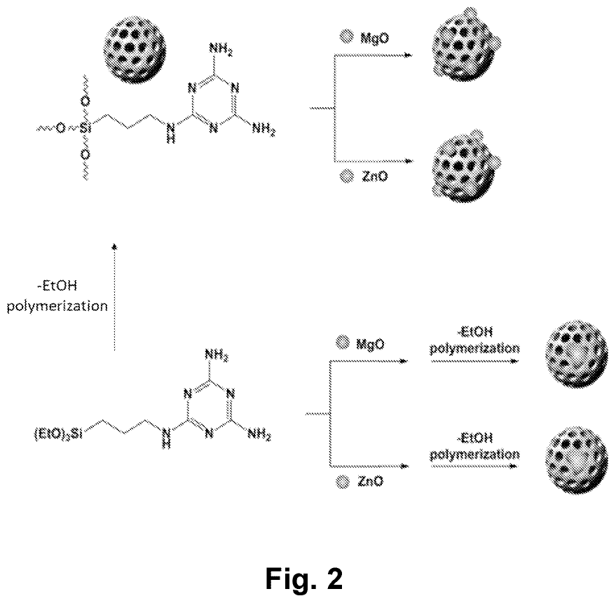

Complexation of metal oxides with silica-melamine can take place either in the presence of the monomer (prior to ethanol removal and polymerization) or in the presence of the final polymer (before halogenation). When the silica-melamine polymer is reacted with the metal oxide, said metal oxide is concentrated rather on the surface of the polymer particles; when the silica-melamine monomer is reacted with the metal oxide and only then polymerized, said metal oxide is rather encapsulated within the polymer particles. FIG. 2 shows schematically both mentioned embodiments of the process of the invention, for specific cases when the monomer is melamine propyltriethoxysilicate, and said metal is magnesium or zinc.

In one embodiment, the halogenation step comprises chlorination of the melamine part of the adduct, carried out at room temperature in aqueous hypochlorite at mildly acidic pH.

The adducts are powders insoluble in water; for examining their biocidal activity they are, in one alternative, suspended in a solution of poly(ethylene-co-vinyl acetate) (PEVA) polymer in toluene (PEVA dissolved in toluene at 50° C. and cooled to 30° C., 15-20% polymer final content).

The invention will be further described and illustrated by the following examples.

### Examples

### Chemicals

Chemicals were obtained from Merck, formerly Sigma Aldrich.

### Analytical Methods

The adducts were characterized by FTIR-ATR (Thermo scientific), SEM-EDAX (Pro, Phenom), and TEM (FEI). The spectra were recorded and compared to starting materials.

### Synthetic Procedures

### Triethoxypropylsilyl Melamine

3-aminopropyl triethyoxysilane, 1 ml, was reacted with 2-chloro-4,6-diamino-1,3,5-triazine, 0.625 g, and bicarbonate, 0.44 g, in 25 ml of ethanol/water 1:1, at 90° C., for 8 hr, under magnetic stirring (see FIG. 1A). A clear solution was obtained.

Poly(propylsilyl-melamine)

The above clear solution was evaporated under reduced pressure at 40° C. until the ethanol was distilled. The resulting polymer precipitated from the water phase and was collected by filtration (see FIG. 1A). The solid product of poly(propylsilyl-melamine) was washed with water and dried under reduced pressure.

Chlorinated poly(propylsilyl-melamine)

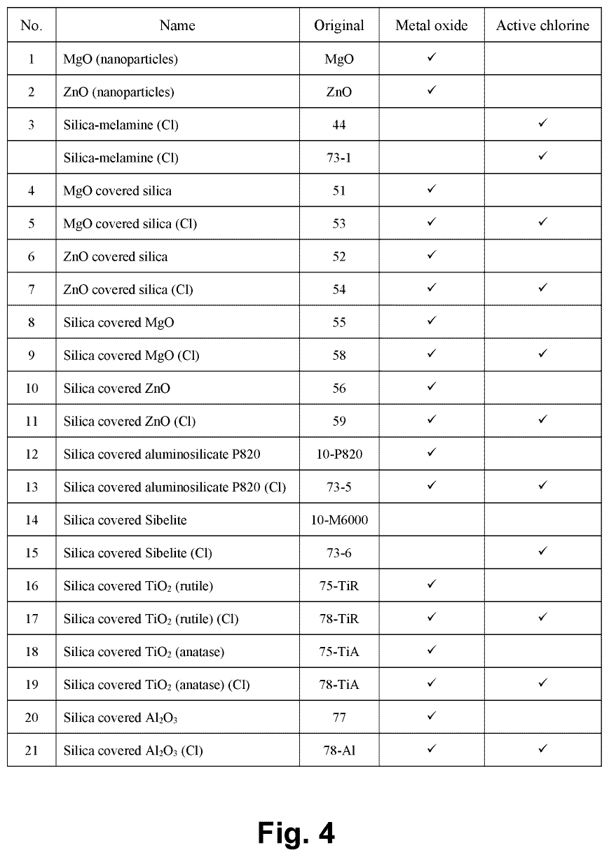

Poly(propylsilyl-melamine), ^{˜}1 g, was dispersed in 3 ml water and acidified with 0.5 ml acetic acid. 5% NaOCl, 35 ml, was added in portions with strong magnetic stirring, while maintaining pH=6 with acetic acid. The resulting slurry was mixed at RT for 2-3 hours (see FIG. 1A). The solid product of chlorinated poly(propylsilyl-melamine) was separated by filtration, washed with water and dried under reduced pressure without exceeding 35° C. See product 44 in the table of FIG. 4.

Poly(propylsilyl-melamine) Metal Oxide Complex (Polymerized and then Mg or Zn Complexed)

Poly(propylsilyl-melamine), 1 g, was mixed with MgO or ZnO, 1 g, respectively, in 13 ml ethanol and 10 ml water and sonicated for 30 min. The reaction was heated at 95° C. for 8 hr under magnetic stirring. The solid product, with MgO or ZnO, was then separated by filtration and dried under reduced pressure. See FIG. 2 and products 51 and 52 in FIG. 4, respectively (“MgO covered silica”, or “ZnO covered silica”).

Poly(propylsilyl-melamine) Metal Oxide Complex (Mg or Zn Complexed and then Polymerized)

Triethoxypropylsilyl melamine, 1 g, was mixed with MgO or ZnO, 1 g, respectively, at 90° C. for 2 hr under magnetic stirring. The reaction mixture was then evaporated under reduced pressure at 40° C. until all the ethanol distilled out. The solid product, with MgO or ZnO, was separated by filtration and dried under reduced pressure. See FIG. 2 and products 55 and 56 in FIG. 4, respectively (“silica-covered MgO” or “silica-covered ZnO”).

Chlorinated poly(propylsilyl-melamine) Metal Oxide (Polymerized and then Mg or Zn Complexed)

Products 51 or 52, respectively, ^{˜}1 g, were dispersed in 3 ml water and acidified with 0.4 ml acetic acid. 5% NaOCl, 18 ml, was added in portions with strong magnetic stirring while maintaining pH=6 with acetic acid. The resulting slurry was mixed at RT for 2-3 hours (see FIG. 1A). The solid product of chlorinated poly(propylsilyl-melamine) was separated by filtration, washed with water and dried under reduced pressure without exceeding 35° C. See FIG. 2 and products 53 and 54 in FIG. 4 (chlorinated “MgO covered silica” or “ZnO covered silica”).

Chlorinated poly(propylsilyl-melamine) Metal Oxide (Mg or Zn Complexed and then Polymerized)

Products 55 or 56, respectively, ^{˜}1 g, were dispersed in 3 ml water and acidified with 0.4 ml acetic acid. 5% NaOCl, 18 ml, was added in portions with strong magnetic stirring, while maintaining pH=6 with acetic acid. The resulting slurry was mixed at RT for 2-3 hours (see FIG. 1A). The solid product of chlorinated poly(propylsilyl-melamine) was separated by filtration, washed with water and dried under reduced pressure without exceeding 35° C. The chlorinated solid product, with MgO or ZnO, was separated. See FIG. 2 and products 58 and 59 in FIG. 4, respectively (chlorinated “Silica-covered MgO” or “Silica-covered ZnO”).

Nonchlorinated and Chlorinated poly(propylsilyl-melamine) Metal Oxide (Al or Ti) Complexed and then Polymerized)

Analogously to products 55, 58, 56, and 59, aluminosilicate or sibelite were employed, and products 10-P820, 73-5, 10-M6000, and 73-6 in FIG. 4, respectively, were obtained.

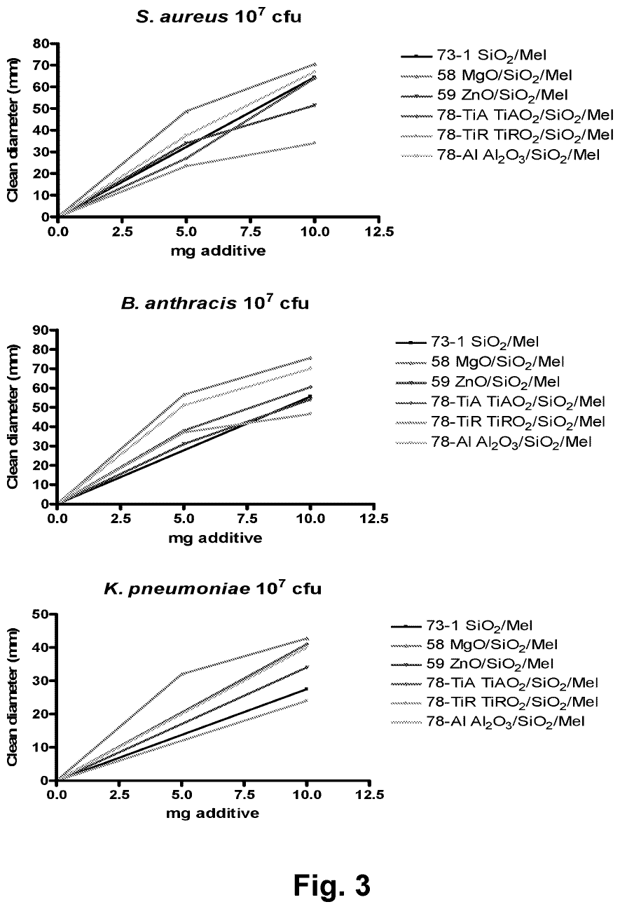

Similarly, the preparation of titanium oxide or aluminum oxide complexes included mixing with anatase or rutile (for Ti), or alumina (for Al), followed by polymerization and chlorination (see FIG. 3 to FIG. 5).

### Antimicrobial Tests

In one group of tests, the tested materials in desired amounts were suspended in a 20% mixture (0.7 ml per disc) of poly(ethylene-co-vinyl acetate) (PEVA) in toluene at 30° C., cast into rectangular or round molds, and cooled to room temperature. Toluene is allowed to evaporate in a hood overnight and solid films were obtained, with the additives dispersed within PEVA.

Polymer films were tested for biocidal activity in two modes:

1. AATCC 147-2016 method, where a rectangular film was placed over 5 lines drawn in tryptose agar with a single dip in bacterial suspension. This method is usually used to test antibacterial fabrics, by measuring clean zone beneath the sample and around it.

2. A specific amount of bacteria (usually 10⁷ cfu) was used to evenly contaminate a tryptose agar plate. A round polymer disc was placed in the middle and a clean zone was measured. This method is used in the determination of antibiotic efficacy, and was used here to measure the relation between adduct amount and biocidal effect (clean zone diameter).

The clean zone diameter caused by PEVA disks on agars were measured. Amounts of either 5 mg (materials without oxide) or 10 mg (materials with oxide, 1:1) were employed. The antibacterial activity was tested with three bacteria: Bacillus anthracis ATCC 14578ΔpXO1ΔpXO2 (Gram-positive, spores), Staphylococcus aureus ATCC 29213 (Gram-positive), and Klebsiella pneumoniae ATCC 10031 (Gram-negative).

B. anthracis, S. aureus, and K. pneumoniae were employed for examining the antibacterial activities of some raw materials and several synthesized melamine-silicate derivatives. FIG. 3 shows the dose dependent pesticidal activity of the examined materials, as secreted from PEVA disks. Melamine-silicate complexed with magnesium oxide, then polymerized and chlorinated (compound 58) showed the highest activity, followed by melamine-silicate complexed with aluminum oxide and titanium oxide, then polymerized and chlorinated (compounds 78-Al and 78-TiA, respectively).

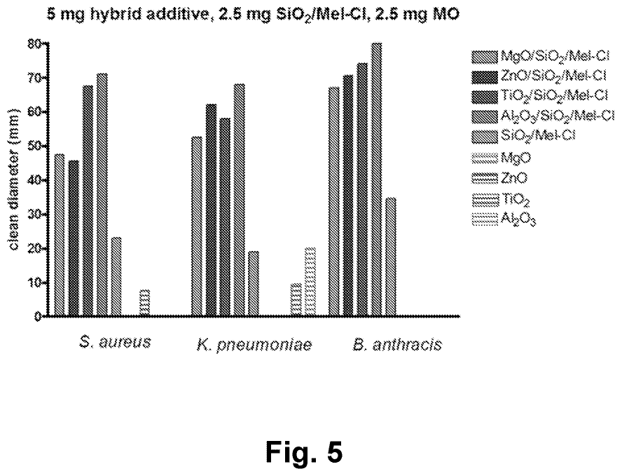

Antimicrobial tests without polymer vehicle were also employed: tryptose agar plates were treated with 10⁷ cfu of bacteria. Holes were punched in the agar, and materials in the shown amounts were applied and incubated at 37° C. for 24 h, and then the diameters of the clean zones around the holes were evaluated. FIG. 5 shows the pesticidal activity of the examined materials (as reflected by the clean zone diameters), obtained by complexing triethoxypropylsilyl melamine with either no metal oxide, or magnesium oxide, or zinc oxide, or titanium oxide of the anatase type, or titanium oxide of the rutile type, or aluminum oxide, followed by polymerizing and chlorinating, as described above.

While the invention has been described using some specific examples, many modifications and variations are possible. It is therefore understood that the invention is not intended to be limited in any way, other than by the scope of the appended claims.

1. A biocidal disinfection and decontamination material comprising halogenated adduct of a metal oxide with a poly(melamine-silicate), having the following structure: 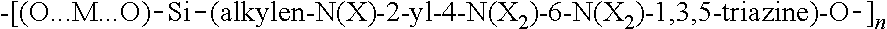
wherein O . . . M . . . O symbolizes adduct of metal oxide with silicate,
the bracketed member [( )Si-( )O—]ₙ stands for n-times repeated unit of substituted silica in the polymer, X stands for halogen, and M stands for metal,
and wherein said alkylene is selected from C2 to C8-alkylene, and said metal is selected from Mg, Zn, Ca, Fe, Cu, Ti, Ag, and Al.

2. The biocidal disinfection and decontamination material of claim 1, wherein said halogenated adduct has the following structure: 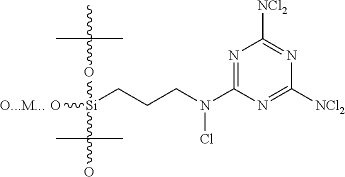
wherein said metal is selected from Mg, Zn, Ca, Fe, Cu, Ti, Ag, and Al.

3. An adduct of metal oxide with a poly(melamine-silicate), having the following structure: 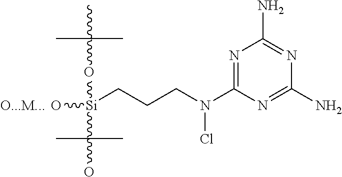
where M is selected from the group consisting of Mg, Zn, Ca, Fe, Cu, Ti, Ag, and Al, for preparing the biocidal disinfection and decontamination material of claim 2, wherein said adduct is stable on storage and can be converted to said biocidal disinfection and decontamination material when needed by chlorinating with NaOCl.

4. A process for manufacturing the biocidal disinfection and decontamination material of claim 1, comprising steps of:
i) reacting ω-aminoalkyl trialkoxysilane with 2-chloro-4,6-diamino-1,3,5-triazine, thereby providing a melamine-silicate based monomer of the following structure:
2-NH-alkyltrialkoxysilyl-4-NH₂-6-NH₂-1,3,5-triazine
wherein said alkyl is selected from C2 to C8-alkyl, and said alkoxy is selected from C1 to C4-alkoxy;
ii) complexing said monomer with said a metal oxide and then polymerizing said monomer, or polymerizing said monomer and then complexing the product with said metal oxide, the two ways providing two different adducts with different properties; and
iii) halogenating the adduct of step ii) with NaOCl or bromine.

5. The process of claim 4, comprising steps of:
i) reacting 3-aminopropyl triethoxysilane with 2-chloro-4,6-diamino-1,3,5-triazine, thereby providing a melamine-silicate based monomer of the following structure: 2-aminoalkyltriethoxysilyl-4,6-diamino-1,3,5-triazine;
ii) complexing said monomer with said metal oxide and then polymerizing said monomer, thereby obtaining a polymer adduct; and
iii) chlorinating the adduct of step ii) with NaOCl.

6. The process of claim 4, comprising steps of:
i) reacting 3-aminopropyl triethoxysilane with 2-chloro-4,6-diamino-1,3,5-triazine, thereby providing a melamine-silicate based monomer of the following structure: 2-aminopropyltriethoxysilyl-4,6-diamino-1,3,5-triazine;
ii) polymerizing said monomer and then complexing the product with said metal oxide, thereby obtaining a polymer adduct; and
iii) chlorinating the adduct of step ii) with NaOCl.

7. The process according to claim 4, wherein said metal oxide is selected from the group consisting of MgO, ZnO, CaO, Fe₂O₃, Cu₂O, CuO, TiO₂, Al₂O₃, Ag₂O, and AgO.

8. The process according to claim 4, said step of polymerization comprising heating and distilling out the released ethanol.

9. A process for efficiently and safely destroying or neutralizing a harmful biological agent on a surface or in a volume, the agent selected from viruses, bacteria, fungi, protozoa, algae, and parasites, comprising contacting said surface or volume with the biocidal material comprising halogenated adduct of a metal oxide with a poly(melamine-silicate) according to claim 1.

10. A process for decontaminating a surface or a volume contaminated with a biological agent sensitive to oxidizers, comprising i) complexing a metal oxide with a melamine-silicate either before or after polymerizing said melamine silicate, ii) halogenating said melamine-silicate metal complex, and iii) contacting said surface or volume with said halogenated complex.

11. The process of claim 10, wherein said halogenating comprises applying NaOCl.

12. The biocidal disinfection and decontamination material prepared according to claim 4 for use in reducing or eliminating the deleterious biological activity of an agent selected from viruses, bacteria, fungi, protozoa, algae, parasites or eggs thereof, and toxic organic agents including peptides, proteins, lipids, polysaccharides, bacterial or fungal or algal toxins.

13. A textile product impregnated with the biocidal disinfection and decontamination material of claim 1, for providing protective dressings, surgical drapes, or surgical clothes and other auxiliary textiles needed during medical treatments.

14. A textile product impregnated with the biocidal disinfection and decontamination material of claim 1, for protecting the skin of a subject against a microbial threat or for supporting healing process in the skin of a subject.

15. The textile product of claim 14, being an active antibacterial dressing for contacting the skin of a subject suffering from skin trauma, such as acute skin injury, chronic skin wound, surgical wound, or chronic skin condition including autoimmune or allergic problems.

16. The textile product of claim 14, being an active dressing for contacting the skin of a subject suffering from acne.

17. A mixture of the biocidal disinfection and decontamination material of claim 1 and gel, ointment, or cream.

18. The mixture of claim 17, for contacting the skin of a subject suffering from a skin condition aggravated by microbial infections, wherein said mixture contacts the skin directly of after being impregnated within a textile material.
